(19) United States
(12) Patent Application Publication
(10) Pub. No.: US 2024/0277918 A1
(43) Pub. Date: Aug. 22, 2024

(54) EXTERNAL SIGNALING SAFEGUARDS FOR INFUSION PUMP

(71) Applicant: B. Braun Medical Inc., Bethlehem, PA (US)

(72) Inventors: Michael Robert Pavlovic, Stroudsburg, PA (US); Vladislav Los, Palmerton, PA (US); James Arnold Thompson, Schnecksville, PA (US)

(73) Assignee: B. Braun Medical Inc., Bethlehem, PA (US)

(21) Appl. No.: 18/111,102

(22) Filed: Feb. 17, 2023

(51) Int. Cl. A61M 5/142; G16H 20/17

(52) U.S. Cl. CPC A61M 5/142; G16H 20/17; A61M 2205/50

(57) ABSTRACT

Multiple hardware and software external signaling safeguards are employed by an infusion pump to ensure proper operation and to prevent the possibility of overinfusion or underinfusion due to unauthorized signals. All communications with the infusion pump pass through a communications module, and a collection of interconnected controller cores process communications within the infusion pump. The communication module provides wireless and wired communication and manages all communications with the controller cores. Communication through the wired communication interface is only permitted when the infusion pump is not actively infusing. When the infusion pump is actively infusing, no communication via the wired communication interface is permitted. The validity of certificates from external wired and wireless signal sources are checked to ensure the source is authorized to communicate with the infusion pump. In addition, all communications are expected to utilize proprietary messaging protocols. Messages received by the infusion pump in other formats are rejected.

### FIELD OF THE INVENTION

The present disclosure is related to infusion pumps and, more particularly, to infusion pumps that use external signaling safeguards to ensure proper operation and to prevent the possibility of overinfusion or underinfusion due to unauthorized external signals.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids are desirable to optimize treatment of a patient as there are many fluids where small variations can be critical.

Infusion pumps are typically setup and programmed by medical professionals in a medical setting. However, ambulatory infusion pumps may be operated by a patient in a home setting. Signaling safeguards are desirable for safe programming and operation of infusion pumps, particularly when the infusion pumps are exposed to a variety of cybersecurity threats and attack vectors.

### SUMMARY

Examples described herein are directed to methods implemented by infusion pumps for delivering fluids to a patient. In sample configurations, multiple hardware and software external signaling safeguards are employed to ensure proper infusion pump operation and to the prevent possibility of overinfusion or underinfusion due to unauthorized signals. All communications with the infusion pump pass through a communications module (“comm module”) and are validated to ensure that the communications are sourced from a known and trusted source before they are passed on to a collection of interconnected controller cores (pump controller core, supervisor controller core, system controller core, and user interface controller core) that process communications within the infusion pump. The comm module is capable of wireless communication (e.g., via WI-FI®/LTE-M) via a wireless interface and wired communication (e.g., via USB) via a wired interface. The wired interface is provided to configure and diagnose the infusion pump and is intended to be used by a technician in conjunction with a provided external software tool. The wireless interface is provided to interact with an enterprise-based software package (either installed on a hospital network or in a cloud system) to not only re-configure the base workings of the infusion pump, but to also provide real-time infusion information to and from the infusion pump. The safeguards provided by the comm module prevent unauthorized access to the infusion pump via the comm module that could interfere with proper operation.

Communication through the wired interface of the comm module is only permitted when the user interface controller core indicates that the infusion pump is not actively infusing. It is, however, possible to connect to an infusion pump via the wireless interface to enable the infusion pump to communicate to provide status information and to receive non-infusion-interrupting autoprogrammed infusions and information for staging software and configuration updates, even when the infusion pump is actively infusing. On the other hand, when the infusion pump is actively infusing, no communication is permitted through the wired interface. This is done to prevent a user from reconfiguring the infusion pump while actively infusing.

The comm module includes a communications application through which wireless and wired communications must pass. The communications application verifies that wireless communications received via the wireless interface are from a valid source, but for the wired communications received via the wired interface, an application on the pump side (user interface controller core) is responsible for validation and verifies the source. The communications application checks certificates from external signal sources to ensure the source is authorized to communicate with the infusion pump and that the certificates are current and have not been revoked. Wireless and wired communication is only possible with a valid certificate. On the other hand, internal communication sources are “trusted” once they are verified and installed properly on the infusion pump.

In addition, all communications received from external sources to the infusion pump are expected to utilize proprietary messaging protocols. If messages are received by the infusion pump in other formats, they are rejected, thus providing another level of signaling protection.

The user interface controller core also has a different operating system than the supervisor controller core, system controller core, and the pump controller core. The use of different operating systems increases the complexity of the system and makes it more difficult for malicious signals to pass due to the need to circumvent the safeguards of both operating systems.

In sample configurations, an infusion pump is described that includes a pump motor configured to pump a liquid through a tube, a power source for the pump motor, at least one pump controller for controlling the pump motor, and a communication module. The communication module includes a communications application that manages communications with the at least one pump controller from external communication sources via wired and wireless communication interfaces. Communication with the at least one pump controller is permitted via the wired interface when the pump motor is not actively infusing liquid through the tube and communication with the at least one pump controller via the wired communication interface from sources external to the infusion pump is blocked when the pump motor is actively infusing liquid through the tube. In sample configurations, the wireless communication interface communicates with the external communication sources to the infusion pump and the wired communication interface communicates with communication sources within and external to the infusion pump. The at least one pump controller may provide status information to and may receive non-infusion-interrupting autoprogrammed infusions and information for staging software and configuration updates from the external communication sources via at least one of the wired or wireless communication interfaces. In the sample configurations, the communications application checks a certificate from an external communication source via the wireless communication interface to check that the certificate is valid and that the external communication source is authorized to communicate with the at least one pump controller. The communications application also checks a messaging protocol of a message received from an external communications source via the wireless communication interface and only permits the message to be received by the at least one pump controller via the wireless communication interface when the messaging protocol is a predetermined proprietary messaging protocol.

In sample configurations, the at least one pump controller includes multiple controller cores including a supervisor controller core, a pump controller core, a system controller core, and a user interface controller core that are collectively adapted to ensure proper operation of the pump motor. The supervisor controller core monitors the other controller cores for improper behavior and stops the pump motor by, for example, cutting power from the power source in the event any one of the other multiple controller cores is determined to be behaving improperly. For example, if an issue is detected on the infusion pump where the pump motor should be stopped, not only may the pump controller core attempt to stop the motor on its own, but the system controller core may also command the pump controller core to stop the motor. If the pump controller core is defective and cannot be communicated with by any other controllers, and the pump motor is still rotating, then the last defense to stop the flow of fluid is to cut the power to the pump motor. The supervisor controller core thus provides a backup to cut power to the pump motor entirely in the case that the pump controller core does not do so. If the pump controller core is able to stop the motor, it will have the proper procedures still in place to “brake” the motor. The pump controller core controls movement of the pump motor and the user interface controller core controls a user interface of the infusion pump. In the sample configurations, the user interface controller core has a different operating system than at least one of the supervisor controller core, the pump controller core, or the system controller core.

The scope of the description provided herein further includes a method of providing signaling safeguards for an infusion pump and a non-transitory computer-readable storage medium that stores controller-executable instructions that, when executed by a communication module of an infusion pump, cause the infusion pump to perform operations including: upon receipt of a message from a communication source within the infusion pump or from an external communication source to the infusion pump, determining whether a pump motor of the infusion pump is actively infusing liquid through a tube, and when the pump motor is actively infusing liquid through the tube, blocking communication with the at least one pump controller via a wired communication interface from external sources.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example cassette with a free flow prevention clamp for use with the ambulatory peristaltic infusion pump of FIG. 1.

FIG. 3 is a partial perspective view of the ambulatory peristaltic infusion pump of FIG. 1 illustrating cams that engage the free flow prevention clamp when the cassette is coupled to the ambulatory peristaltic infusion pump.

FIGS. 4 and 5 are cutaway views of the ambulatory peristaltic infusion pump of FIG. 1 illustrating pump fingers and cams for moving the pump fingers.

FIG. 6 is a diagram illustrating the communication module and multiple controller cores that together ensure proper operation of the ambulatory peristaltic infusion pump in a sample configuration.

FIG. 7 is a flow chart illustrating the combined operation of the communication application of the communication module and software running on the user interface controller core for safe operation of the ambulatory peristaltic infusion pump in a sample configuration.

FIG. 8 is a functional block diagram illustrating a general-purpose computer hardware platform configured to implement the functional examples described with respect to FIGS. 1-7.

FIG. 9 is another functional block diagram illustrating a general-purpose computer hardware platform configured to implement the functional examples described with respect to FIGS. 1-7.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the pump safeguards described herein may be used with a variety of other pump types.

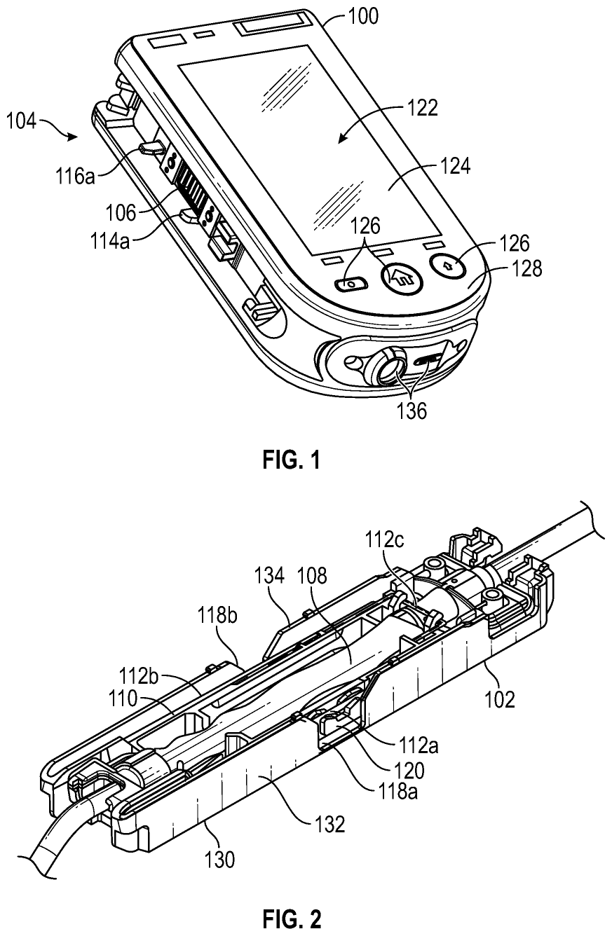

FIG. 1 depicts an example ambulatory peristaltic infusion pump 100, while FIG. 2 depicts an example cassette 102 for use with the ambulatory peristaltic infusion pump 100. The ambulatory peristaltic infusion pump 100 includes a receptacle 104 configured to receive the cassette 102. A peristaltic pump 106 within the receptacle 104 acts upon a tube 108 extending through a channel within the cassette 102 to pump fluid from a fluid container (e.g., a bag or a bottle; not shown) into a patient. An example free flow prevention clamp 110 is positioned within the cassette 102 to allow fluid flow through the tube 108 when the cassette 102 is coupled to the ambulatory peristaltic infusion pump 100 within the receptacle 104 (during which time the peristaltic pump 106 controls fluid flow through the tube 108) and to selectively cut off fluid flow through the tube 108 when the cassette 102 is not coupled to the ambulatory peristaltic infusion pump 100 in order to prevent unintentional fluid flow through the tube 108 (e.g., free flow).

The ambulatory peristaltic infusion pump 100 includes a user interface 122 for interacting with the ambulatory peristaltic infusion pump 100. The illustrated user interface 122 includes a display 124 (which may be a touchscreen) and buttons 126. A user controls operation of the ambulatory peristaltic infusion pump 100 via the user interface 122. The ambulatory peristaltic infusion pump 100 additionally includes a housing 128 for containing and supporting the components of the ambulatory peristaltic infusion pump 100 such as the peristaltic pump 106, electronics, power supplies, and the like.

The free flow prevention clamp 110 includes a first elongate section 112a, a second elongate section 112b, and a clamping section 112c. The housing 130 of the cassette 102 supports the free flow prevention clamp 110. The clamping section 112c is positioned within the cassette geometry such that, when the cassette 102 is received within the receptacle 104 of the ambulatory peristaltic infusion pump 100, the clamping section 112c extends across the channel receiving the tube 108. The housing 130 of the cassette 102 may be rigid plastic or other material capable of supporting the tube 108 and free flow prevention clamp 110.

The ambulatory peristaltic infusion pump 100 also includes a pair of arc cams 114a and 114b. First arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the ambulatory pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

The cassette 102 also includes a first cutout 118a in a sidewall 132 of the cassette 102 and a second cutout 118b in an opposite sidewall 134 of the cassette 102. Additionally, the cassette 102 includes a touch pad 120 positioned on the first elongate section 112a adjacent a mid-point of the first elongate section 112a and the first cutout 118a. The touch pad 120 and cutout 118a together facilitate engagement of the first elongate section 112a by a finger of an operator in order to manually lift the clamping section 112c to allow fluid flow through the tube 108 (e.g., for priming the cassette 102) when the cassette 102 is not received within the receptacle 104 of the ambulatory peristaltic infusion pump 100. The touch pad 120 may be a press fit piece of rigid plastic. Although the touch pad 120 is illustrated as only on the first elongate section 112a, the touch pad 120 also may be provided on the second elongate section 112b.

The ambulatory infusion pump 100 further includes connector ports 136 that provide electronic access for control and for powering the ambulatory infusion pump 100 when used in the configurations described below.

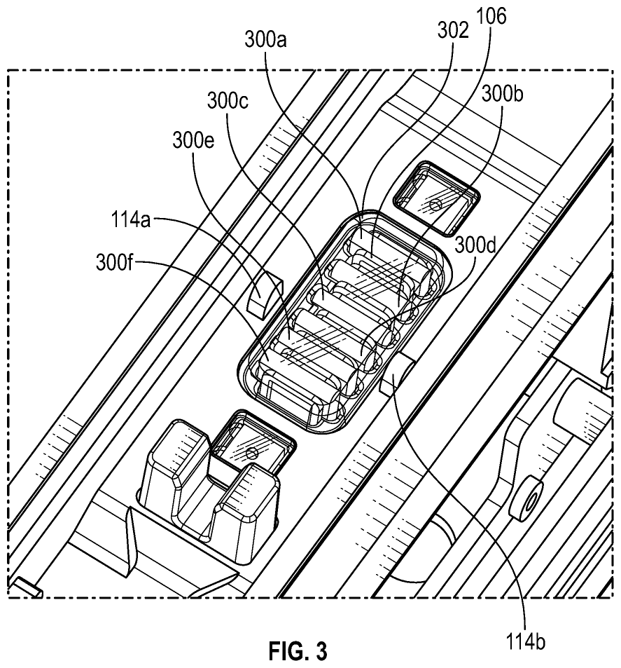

FIG. 3 depicts the arc cams 114a and 114b and peristaltic pump 106 of the ambulatory peristaltic infusion pump 100. The peristaltic pump 106 includes multiple pump fingers 300 (six pump fingers 300a-f illustrated in FIG. 3). A flexible barrier 302 separates the pump fingers 300 (and other pump components of a pumping mechanism) from the receptacle 104 receiving the cassette 102 with the tube 108. The flexible barrier 302 provides a barrier between the fluid delivery apparatus/cassette and the pumping mechanism to prevent fluid from damaging components of the pumping mechanism.

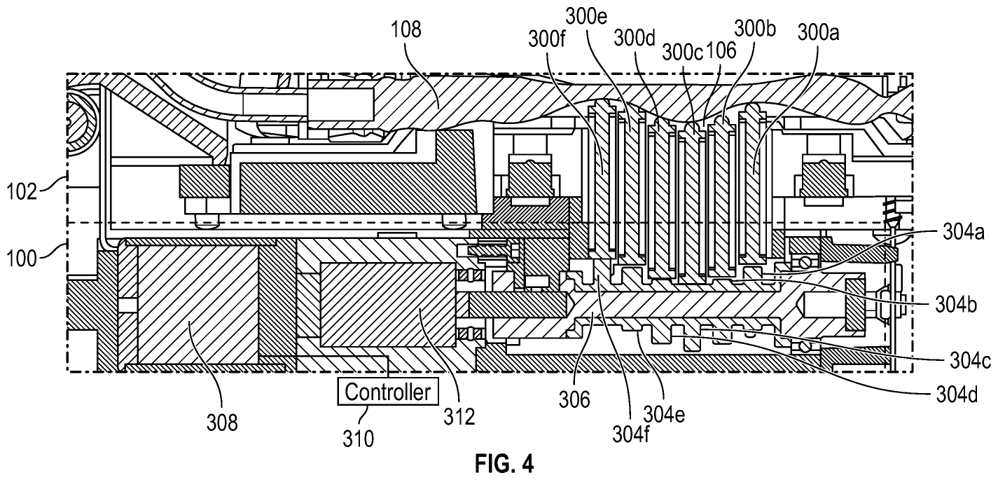

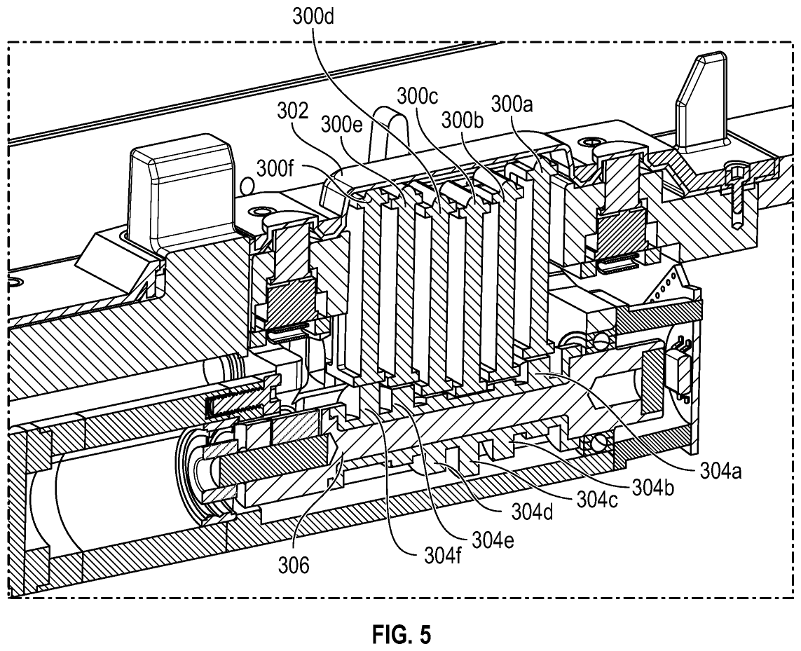

FIGS. 4 and 5 are cutaway views of the ambulatory peristaltic infusion pump 100 with the cassette 102 inserted into the receptacle 104 of the ambulatory infusion pump 100. Multiple cams 304 (six cams 304a-f) supported by a camshaft 306 act on respective pump fingers 300a-300f. The cams 304a-304f respectively raise and lower the pump fingers 300a-300f, which engage the tube 108 of the cassette 102 in order to force fluid though the tube 108. A pump motor 308 under control of a pump controller 310 turns the camshaft 306 by way of a gearbox 312. As the camshaft 306 turns, the cams 304a-300f, which are offset from each other in an axial direction, raise and lower respective pump fingers 300a-300f. For example, cam 304a raises and lowers pump finger 300a; cam 304b raises and lowers pump finger 300b, and the like. The pump controller 310 may be a standalone or embedded processor configured to carry out instructions in order to control operations of the ambulatory peristaltic infusion pump 100.

The pump controller 310 may include a user interface controller core/system controller core such as a dual core 32 bit processor from NXP of Eindhoven, Netherlands (e.g., model #MCIMX7S5EVM08SC), a pump controller core from NXP (e.g., model #MKV31F512VLH12), a supervisor controller core from NXP (e.g., model #MKL17Z128VMP4), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion rate from a system controller core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 306 of the pumping mechanism is measured by the magnetic encoder. At specified time intervals, the output of the encoder is read by the microcontroller, which uses the encoder value to compute the speed of the camshaft 306 and the position of the pump rotation. These values are then used to modify the PWM output to maintain the correct camshaft RPM.

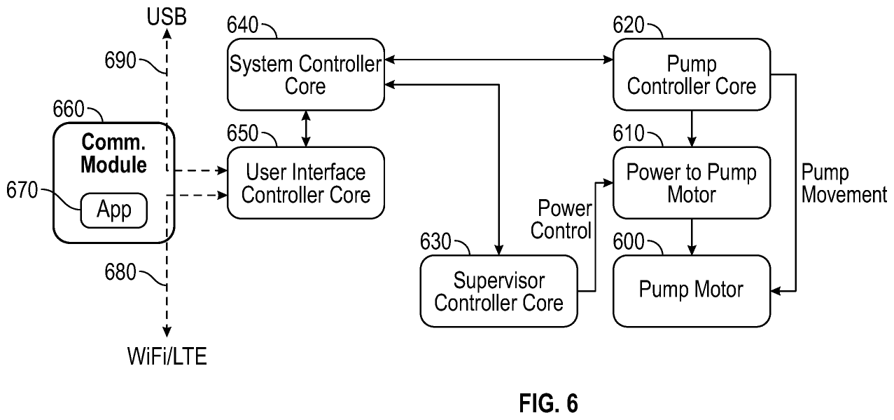

In sample configurations, the pump controller 310 includes multiple cores designed to ensure safe operation of the ambulatory peristaltic infusion pump 100. As described above with respect to FIGS. 1-5, the ambulatory peristaltic infusion pump 100 includes a peristaltic pump 106 configured to deliver fluid (e.g., pain medication) to a patient in response to pump controller 310. FIG. 6 is a diagram illustrating the communication module 660 and multiple controller cores 620-650 used as pump controller 310 that together ensure safe operation of the peristaltic pump 106 in sample configurations. As shown in FIG. 6, the peristaltic pump 106 includes a pump motor 600 that is powered by a power source 610 and controlled by a collection of interconnected controller cores 620-650 that monitor operation of each other and process communications within the peristaltic pump 106.

In addition, multiple hardware and software external signaling safeguards are employed to ensure proper operation of the ambulatory peristaltic infusion pump 100 and to prevent the possibility of overinfusion (or underinfusion) due to unauthorized signals. For this purpose, all communications with the ambulatory peristaltic infusion pump 100 pass through a communications module (“comm module”) 660 that processes communications within the ambulatory peristaltic infusion pump 100. The comm module 660 is capable of wireless communication and wired communication. In addition to such hardware safeguards, software safeguards are implemented by a communications application (“comm app”) 670 as well as pump side applications that validate the USB communication path to prevent unauthorized access to the ambulatory peristaltic infusion pump 100 via the comm module 660 that could interfere with proper operation.

In the configuration illustrated in FIG. 6, the pump controller core 620 is dedicated to control movement of the pump motor 600. The pump controller core 620 may be located on a circuit board to which the pump motor 600 is electrically connected. The pump core controller 620 is responsible for controlling movement of the pump motor 600 (e.g., start/stop/rotation rate/etc.). No other controller core has direct access to the pump motor 600 (with the limited exception of the supervisor controller core 630). The power to pump motor 600 is provided by a pump motor driver (not shown). The supervisor controller core 630 controls overall power to the pump motor driver, but the pump controller core 620 also has the ability to enable/disable the pump motor driver, which in turn provides the power to the pump motor 600.

The supervisor controller core 630 is dedicated to supervisory operations for the pump motor 600. The supervisor controller core 630 can stop the pump motor 600 by, for example, controlling the power to the pump motor at power source 610 in the event any one of the other controller cores 620, 640, or 650 is determined to be behaving improperly. The supervisor controller core 630 monitors the health of all other controller cores in the ambulatory peristaltic infusion pump 100. In the event that a controller core is behaving improperly, the supervisor controller core 630 is capable of stopping power flow to the pump motor 600 by means of switching off the power source 610 to the pump motor 600. It will be appreciated that the pump motor 600 may be stopped without cutting all power to the pump motor 600.

The system controller core 640 and user interface controller core 650 control other operations of the ambulatory peristaltic infusion pump 100 (e.g., graphical user input/output, infusion state logic, alarms, configurations, etc.). Controller cores 640 and 650 are interconnected with each other. The system controller core 640 is interconnected with the pump controller core 620 and the supervisor controller core 630. As noted above, if not explicitly notified of a problem, the supervisor controller core 630 monitors the other controller cores for improper behavior and acts as appropriate.

The comm module 660 includes the comm app 670 that implements a wireless interface 680 and wired interface 690 through which wireless and wired communications must pass. The comm app 670 checks certificates from external wireless signal sources that use the wireless communications interface 680 to ensure the external signal source is authorized to communicate with the ambulatory peristaltic infusion pump 100 and that the certificates are valid (e.g., current and have not been revoked). Pump applications check the certificates on the wired interface path. Communications with the ambulatory peristaltic infusion pump 100 are only possible with a valid certificate. Messages from the wired interface 690 also may be checked for valid certificates, but internal messages from known sources within the ambulatory peristaltic infusion pump 100 need not require such certificates provided physical access to the controller hardware is prevented.

Communications through the wired interface 690 of the comm module 660 is only permitted when the user interface controller core 650 indicates that the ambulatory peristaltic infusion pump 100 is not actively infusing. However, it is possible to connect the ambulatory peristaltic infusion pump 100 to an external source via the wireless interface 690 to enable the ambulatory peristaltic infusion pump 100 to communicate with the external source to provide status information, to receive non-infusion-interrupting information for staging software and configuration updates, and to receive autoprogrammed infusions. On the other hand, when the ambulatory peristaltic infusion pump 100 is actively infusing, no communication is permitted through the wired interface 690 to prevent a user from reconfiguring the ambulatory peristaltic infusion pump 100 during operation. It is noted that any autoprogramming information or staged software and configuration update information received via the wireless interface 680 during an active infusion will not be applied during the current active infusion.

As a further signaling safeguard, all communications received by the ambulatory peristaltic infusion pump 100 from external signal sources are expected to utilize proprietary messaging protocols. If messages are received by the ambulatory peristaltic infusion pump 100 in any other format besides the proprietary messaging protocol, such messages are rejected, thus providing another level of signaling protection.

In addition, in sample configurations, the user interface controller core 650 may have a different operating system than the supervisor controller core 630, the pump controller core 620, the system controller core 640, and/or any other controller core. The use of different operating systems increases the complexity of the pump controller 310 so as to make it more difficult for malicious signals to pass due to the need to circumvent the safeguards of two or more operating systems.

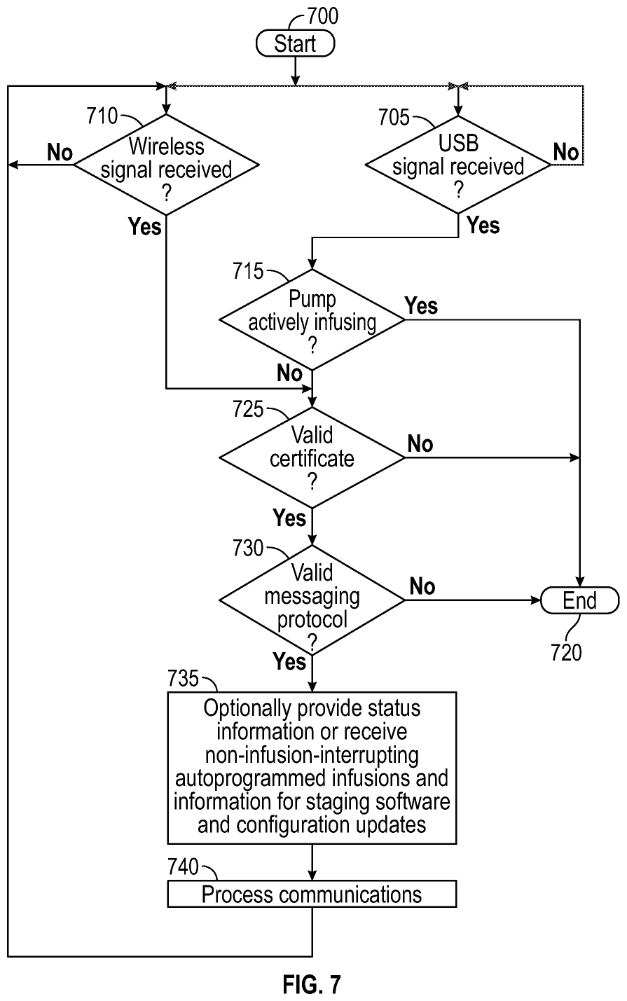

FIG. 7 is a flow chart illustrating the combined operation of the comm app 670 of the comm module 660 and software running on the user interface controller core 650 for safe operation of the ambulatory peristaltic infusion pump 100 in a sample configuration.

As illustrated in FIG. 7, the comm app 670 starts at 700 and waits for receipt of an inbound communication signal via the wired interface 690 at 705 or the wireless interface 680 at 710. Upon receipt of an inbound wired communication signal, software running on the interface controller core 650 checks at 715 whether the ambulatory peristaltic infusion pump 100 is actively infusing. If so, the software running on the interface controller core 650 will close any active communication links that may be open across the wired communication interface, and the process ends at 720. If the ambulatory peristaltic infusion pump 100 is not actively infusing or if the communications are received via the wireless interface 680, communications are permitted. Accordingly, the comm app 670 checks at 725 whether the received message came from a source with a valid certificate if the communications are over a wireless connection. Otherwise, if the message is received over a wired connection, then software running on the user interface controller core 640 checks at 725 if the received message came in from a source that has a valid certificate. If no valid certificate is received, the process ends at 720. However, if a valid certificate has been received, the comm app 670 checks at 730 whether the received message has a valid messaging protocol. If not, the process ends at 720. If the received message has a valid messaging protocol, communications are enabled. The comm app 670 also may initiate outbound communications in order to report status information at 735 to other elements via the wired interface 690 or the wireless interface 680. The comm app 670 also may receive from external sources non-infusion-interrupting autoprogrammed infusions and information for staging software and configuration updates via the wireless interface 680 at 735.

Authorized communications are processed by the user interface controller core 650 at 740. The process repeats for each received message.

Though FIG. 7 illustrates that communications received from external sources via the wired interface 690 are also checked for a valid certificate and a valid messaging protocol, it will be appreciated by those skilled in the art that these steps may be skipped for messages received via the wired interface 690 from internal (i.e., known trustworthy) elements communicating with each other within the ambulatory peristaltic infusion pump 100. Also, limited internal pump communications may be permitted when the ambulatory peristaltic infusion pump 100 is actively infusing.

By separating the functionality of the pump controller 310 and providing signaling safeguards as described herein, operational control safeguards may be maintained whereby the pump motor 600 is immediately stopped upon detection of improper behavior in any of the controller cores 620-650. Also, the hardware and software signaling safeguards described herein prevent unauthorized access to the pump controller 310 by potentially malicious signals from unauthorized parties or by viable signals interrupting ongoing infusions. These safeguards promote safe operation of the ambulatory peristaltic infusion pump 100 described herein as well as other types of infusion pumps known to those skilled in the art.

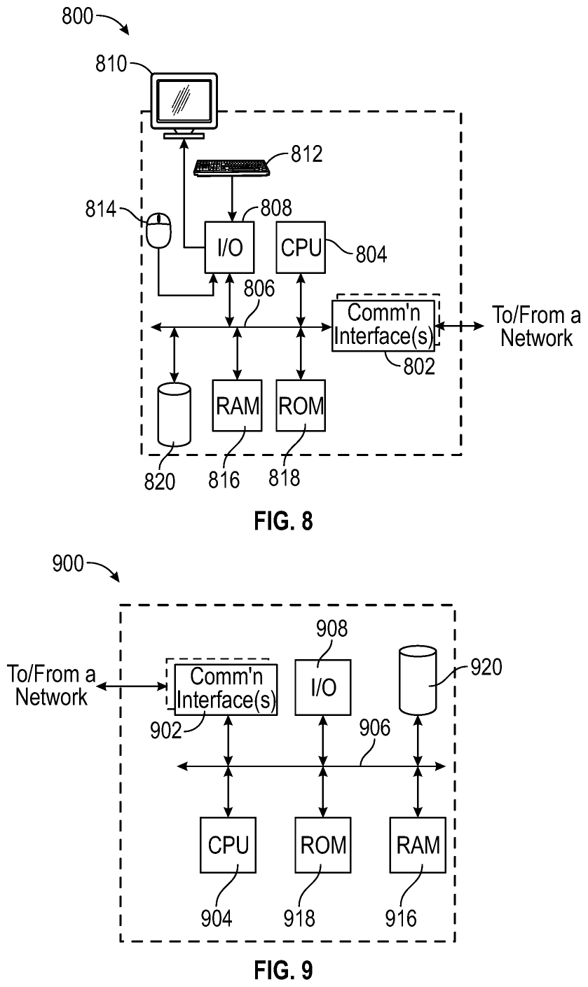

FIGS. 8 and 9 are functional block diagrams illustrating general-purpose computer hardware platforms configured to implement the functional examples described with respect to FIGS. 1-6 as discussed above.

Specifically, FIG. 8 illustrates an example computer platform 800 and FIG. 9 depicts an example computer 900 with user interface elements, as may be used to implement in a personal computer, ambulatory peristaltic infusion pump 100, or other type of workstation or terminal device. It is believed that those skilled in the art are familiar with the structure, programming and general operation of such computer equipment and as a result the drawings should be self-explanatory.

Hardware of an example computer platform 800 (FIG. 8) includes a data communication interface 802 for packet data communication. The computer platform 800 also includes a central processing unit (CPU) 804, in the form of circuitry forming one or more processors, for executing program instructions. The hardware of computer platform 800 typically includes an internal communication bus 806, program and/or data storage 816, 818, and 820 for various programs and data files to be processed and/or communicated by the computer platform 800, although the computer platform 800 often receives programming and data via network communications. In one example, as shown in FIG. 8, the computer platform 800 further includes a video display unit 810 (e.g., a liquid crystal display (LCD) or a cathode ray tube (CRT)), an alphanumeric input device 812 (e.g., a keyboard), a cursor control device 814 (e.g., a mouse), each of which communicate with the internal communication bus 806 via an input/output device (I/O) 808. The hardware elements, operating systems and programming languages of such computer platforms 800 are conventional in nature, and it is presumed that those skilled in the art are adequately familiar therewith. The computer platform 800 may function as a server that may be implemented in a distributed fashion on a number of similar hardware platforms to distribute the processing load.

As illustrated in FIG. 9, hardware of a computer type user terminal device 900, such as a PC or tablet computer, similarly includes a data communication interface 902, CPU 904, main memory 916 and 918, one or more mass storage devices 920 for storing user data and the various executable programs, an internal communication bus 906, and an input/output device (I/O) 908.

Aspects of the methods for pump control, as outlined above, may be embodied in programming in general purpose computer hardware platforms (such as described above with respect to FIGS. 8 and 9), e.g. in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another, for example, from a processor 804 of the system 800 and/or from a pump controller 310 of a peristaltic infusion pump 100 to a computer or software of another system (not shown). Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables, copper wire and fiber optics, including the wires that include a bus within a computer system. Carrier-wave transmission media can take the form of electric or electromagnetic signals or acoustic or light waves such as those generated during radio frequency (RF) and light-based data communications. Common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render the computer system or device into a special-purpose machine that is customized to perform the operations specified in the program performed by the pump controller 310 of the pump 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump, comprising:
a pump motor configured to pump a liquid through a tube;
a power source for the pump motor;
at least one pump controller for controlling the pump motor; and
a communication module including a communications application that manages communications with the at least one pump controller from external communication sources via wired and wireless communication interfaces,
wherein communication with the at least one pump controller is permitted via the wired interface when the pump motor is not actively infusing liquid through the tube and communication with the at least one pump controller via the wired communication interface from sources external to the infusion pump is blocked when the pump motor is actively infusing liquid through the tube.

2. The infusion pump of claim 1, wherein the wireless communication interface communicates with the external communication sources to the infusion pump and the wired communication interface communicates with communication sources within and external to the infusion pump.

3. The infusion pump of claim 1, wherein the at least one pump controller provides status information to and receives non-infusion-interrupting autoprogrammed infusions and information for staging software and configuration updates from the external communication sources via at least one of the wired or wireless communication interfaces.

4. The infusion pump of claim 1, wherein the communications application checks a certificate from an external communication source via the wireless communication interface to check that the certificate is valid and only permits a message to be received from the external communications source via the wireless communication interface when the certificate is valid.

5. The infusion pump of claim 1, wherein the communications application checks a messaging protocol of a message received from the external communications source via the wireless communication interface and only permits the message to be received by the at least one pump controller via the wireless communication interface when the messaging protocol is a predetermined proprietary messaging protocol.

6. The infusion pump of claim 1, wherein the at least one pump controller comprises multiple controller cores including a supervisor controller core, a pump controller core, a system controller core, and a user interface controller core that are collectively configured to ensure proper operation of the pump motor, wherein the supervisor controller core monitors the other controller cores for improper behavior and stops the pump motor from the power source in the event any one of the other multiple controller cores is determined to be behaving improperly.

7. The infusion pump of claim 6, wherein the pump controller core controls movement of the pump motor and the user interface controller core controls a user interface of the infusion pump, and wherein the user interface controller core has a different operating system than at least one of the supervisor controller core, the pump controller core, or the system controller core.

8. A method of providing signaling safeguards for an infusion pump including a pump motor configured to pump a liquid through a tube, a power source for the pump motor, at least one pump controller for controlling the pump motor, and a communication module that manages communications with the at least one pump controller from communication sources within the infusion pump and external communication sources to the infusion pump, the method comprising:
upon receipt of a message from a communication source within the infusion pump or from an external communication source to the infusion pump, determining whether the pump motor is actively infusing liquid through the tube; and
when the pump motor is actively infusing liquid through the tube, blocking communication with the at least one pump controller via a wired communication interface from sources external to the infusion pump.

9. The method of claim 8, wherein blocking communication with the at least one pump controller comprises checking a certificate from an external communication source to determine whether the certificate is valid, and blocking communication with the at least one pump controller when the certificate is not valid.

10. The method of claim 8, wherein blocking communication with the at least one pump controller comprises checking a messaging protocol of a message received from the external communications source and only permitting the message to be received by the at least one pump controller when the messaging protocol is a predetermined proprietary messaging protocol.

11. The method of claim 8, wherein the at least one pump controller comprises multiple controller cores including a supervisor controller core, a pump controller core, a system controller core, and a user interface controller core that are collectively configured to ensure proper operation of the pump motor, wherein the supervisor controller core monitors the other controller cores for improper behavior and stops the pump motor from the power source in the event any one of the other multiple controller cores is determined to be behaving improperly.

12. The method of claim 11, wherein the pump controller core controls movement of the pump motor and the user interface controller core controls a user interface of the infusion pump, further comprising providing a first operating system in the user interface controller core and providing a second operating system in at least one of the supervisor controller core, the pump controller core, or the system controller core.

13. The method of claim 8, further comprising providing status information to and receiving non-infusion-interrupting autoprogrammed infusions and information for staging software and configuration updates from the external communication source via the communication module.

14. A non-transitory controller-readable storage medium storing controller-executable instructions that, when executed by a communication module of an infusion pump cause the infusion pump to perform operations comprising:
upon receipt of a message from a communication source within the infusion pump or from an external communication source to the infusion pump, determining whether a pump motor of the infusion pump is actively infusing liquid through a tube; and
when the pump motor is actively infusing liquid through the tube, blocking communication with at least one pump controller via a wired communication interface from sources external to the infusion pump.

15. The non-transitory controller-readable storage medium of claim 14, wherein the instructions further cause the communication module of the infusion pump to block communication with the at least one pump controller by checking a certificate from an external communication source to determine whether the certificate is valid, and blocking communication with the at least one pump controller when the certificate is not valid.

16. The non-transitory controller-readable storage medium of claim 14, wherein the instructions further cause the communication module of the infusion pump to block communication with the at least one pump controller by checking a messaging protocol of a message received from the external communications source and only permitting the message to be received by the at least one pump controller when the messaging protocol is a predetermined proprietary messaging protocol.

17. The non-transitory controller-readable storage medium of claim 14, wherein the at least one pump controller comprises multiple controller cores including a supervisor controller core, a pump controller core, a system controller core, and a user interface controller core that are collectively configured to ensure proper operation of the pump motor, wherein the supervisor controller core monitors the other controller cores for improper behavior and stops the pump motor in the event any one of the other multiple controller cores is determined to be behaving improperly.

18. The non-transitory controller-readable storage medium of claim 17, wherein the pump controller core controls movement of the pump motor and the user interface controller core controls a user interface of the infusion pump, further comprising executing instructions to provide a first operating system in the user interface controller core and to provide a second operating system in at least one of the supervisor controller core, the pump controller core, or the system controller core.

19. The non-transitory controller-readable storage medium of claim 14, wherein the instructions further cause the communication module to provide status information of the infusion pump to and receive non-infusion-interrupting autoprogrammed infusions and information for staging software and configuration updates from the external communication source.
